SEQUENCE LISTING (8 sequences)

SEQ ID NO: 1 - protein, 70 aa, ARTIFICIAL SEQUENCE
  SYNTHETIC PEPTIDE
  MISC_FEATURE (1)..(10): X IS ANY ONE AMINO ACID, AND ONE OR MORE X MAY BE ABSENT
  MISC_FEATURE (12)..(16): X IS ANY ONE AMINO ACID, AND ONE OR MORE X MAY BE ABSENT
  MISC_FEATURE (18)..(27): X IS ANY ONE AMINO ACID, AND ONE OR MORE X MAY BE ABSENT
  MISC_FEATURE (29)..(38): X IS ANY ONE AMINO ACID, AND ONE OR MORE X MAY BE ABSENT
  MISC_FEATURE (40)..(43): X IS ANY ONE AMINO ACID, AND ONE OR MORE X MAY BE ABSENT
  MISC_FEATURE (45)..(59): X IS ANY ONE AMINO ACID, AND ONE OR MORE X MAY BE ABSENT
  MISC_FEATURE (61)..(70): X IS ANY ONE AMINO ACID, AND ONE OR MORE X MAY BE ABSENT
XXXXXXXXXXCXXXXXCXXXXXXXXXXCXXXXXXXXXXCXXXXCXXXXXXXXXXXXXXXCXXXXXXXXXX

SEQ ID NO: 2 - protein, 7 aa, ARTIFICIAL SEQUENCE
  SYNTHETIC PEPTIDE
  MISC_FEATURE (1)..(1): X is an amino acid chosen from alanine, serine, glycine and threonine
  MISC_FEATURE (2)..(2): X is proline
  MISC_FEATURE (6)..(6): X is proline
  MISC_FEATURE (7)..(7): X is proline
  MISC_FEATURE (3)..(3): X is an amino acid chosen from phenylalanine, tryptophan and tyrosine
  MISC_FEATURE (4)..(4): X is an amino acid chosen from aspartic acid or glutamic acid
  MISC_FEATURE (5)..(5): X is an amino acid chosen from valine, leucine, isoleucine and methionine
XXXXXXX

SEQ ID NO: 3 - protein, 4 aa, ARTIFICIAL SEQUENCE
  SYNTHETIC PEPTIDE
  MISC_FEATURE (2)..(2): X is an amino acid chosen from alanine, serine, glycine and threonine
  MISC_FEATURE (4)..(4): X is an amino acid chosen from alanine, serine, glycine, threonine, aspartic acid and glutamic acid
  MISC_FEATURE (3)..(3): X is an amino acid chosen from alanine, serine, glycine, threonine and a basic residue
  MISC_FEATURE (1)..(1): X is an amino acid chosen from alanine, serine, glycine, threonine and a basic residue
XXXX

SEQ ID NO: 4 - protein, 9 aa, ARTIFICIAL SEQUENCE
  SYNTHETIC PEPTIDE
  MISC_FEATURE (1)..(1): X is an amino acid chosen from valine, leucine, isoleucine and methionine
  MISC_FEATURE (3)..(3): X is an amino acid chosen from valine, leucine, isoleucine and methionine
  MISC_FEATURE (2)..(2): X is proline
  MISC_FEATURE (4)..(4): X is an amino acid chosen from alanine, serine, glycine and threonine
  MISC_FEATURE (8)..(8): X is an amino acid chosen from alanine, serine, glycine and threonine
  MISC_FEATURE (6)..(6): X is an amino acid chosen from valine, leucine, isoleucine, methionine, phenylalanine, tryptophan and tyrosine
  MISC_FEATURE (9)..(9): X is an amino acid chosen from phenylalanine, tryptophan and tyrosine
  MISC_FEATURE (5)..(5): X is an amino acid chosen from valine, leucine, isoleucine and methionine
  MISC_FEATURE (7)..(7): X is an amino acid chosen from valine, leucine, isoleucine and methionine
XXXXXXXXX

SEQ ID NO: 5 - protein, 5 aa, ARTIFICIAL SEQUENCE
  SYNTHETIC PEPTIDE
  MISC_FEATURE (1)..(1): X is a basic amino acid or an amino acid chosen from valine, leucine, isoleucine and methionine
  MISC_FEATURE (2)..(2): X is asparagine or glutamine or a basic amino acid
  MISC_FEATURE (3)..(3): X is proline
  MISC_FEATURE (4)..(4): X is an amino acid chosen from alanine, serine, glycine and threonine
  MISC_FEATURE (5)..(5): X is an amino acid chosen from alanine, serine, glycine and threonine
XXXXX

SEQ ID NO: 6 - protein, 37 aa, ARTIFICIAL SEQUENCE
  SYNTHETIC PEPTIDE
ASCNGVCSPFEMPPCGTSACRCIPVGLVIGYCRNPSG

SEQ ID NO: 7 - protein, 37 aa, ARTIFICIAL SEQUENCE
  SYNTHETIC PEPTIDE
ASCNGVCSPFEMPPCGTSACRCIPVGLVVGYCRNPSG

SEQ ID NO: 8 - protein, 37 aa, ARTIFICIAL SEQUENCE
  SYNTHETIC PEPTIDE
ADCNGACSPFEVPPCRSRDCRCVPIGLFVGFCIHPTG

(19) United States
(12) Patent Application Publication
(10) Pub. No.: US 2008/0086786 A1
(43) Pub. Date: Apr. 10, 2008

(54) USE OF POLYPEPTIDE DERIVED FROM A PA1B LEGUME ALBUMEN AS INSECTICIDE

(75) Inventors: Bernard DELOBEL, Villeurbanne (FR); Annie GRENIER, Chassieu (FR); Jacques GUEGUEN, La Chapelle sur Erdre (FR); Eric FERRASSON, Nantes (FR); Mbaiguinam MBAILAO, TD-N'djamena (TD)

Correspondence Address: OBLON, SPIVAK, MCCLELLAND MAIER & NEUSTADT, P.C., 1940 DUKE STREET, ALEXANDRIA, VA, 22314, US

(73) Assignees: Institut National de la Recherche Agronomique, Paris (FR); Institut National des Sciences Appliquees De Lyon, Villeurbanne (FR)

(21) Appl. No.: 11/859,076

(22) Filed: Sep. 21, 2007

Related U.S. Application Data
(63) Continuation of application No. 09/674,496, filed on Jan. 11, 2001.

(30) Foreign Application Priority Data
May 11, 1998 (FR) 98/05877

(51) Int. Cl. A01H 1/00; A01N 65/00

(52) U.S. Cl. 800/279; 514/12; 514/13

(57) ABSTRACT

The invention concerns the use of a polypeptide derived from a PA1b legume albumen as insecticide, particularly for protecting cereal grains against insect pests.

### CROSS REFERENCE TO RELATED APPLICATIONS

The present application is a continuation application U.S. application Ser. No. 09/674,496 filed Jan. 11, 2001, pending, which is a 371 application of PCT/FR99/01085 filed May 7, 1999. The application also claims the benefit of FR 98/05877 filed May 11, 1998.

### FIELD OF THE INVENTION

The present invention relates to insecticidal proteins and to the use thereof for protecting plants, and in particular cereals, their seeds and products derived from them, against insect pests.

### BACKGROUND

Insects which are pests for cereal seeds are to be found in various families, in particular among Coleoptera, Lepidoptera and Homoptera. Among Coleoptera, mention will be made in particular of grain weevils (Sitophilus oryzae, Sitophilus zeamais, Sitophilus granarius), and of Tenebrio spp, Rhyzopertha dominica, Trogoderma spp. and Triboiiurn coNfusum. Among Lepidoptera, mention will be made in particular of Sitotroga cereaieiia and Ephestia kuehnieiia.

Pests for cereal seeds are among the main enemies of the crops which they attack in the field (at least in hot regions), and especially in storage silos; they may also attack transformed products which are derived from cereals (for example, flours, semolinas, etc). These insects cause very significant damage and, each year, cause the destruction of a large portion(which can come close to 25%) of the world harvest of cereals harvested each year.

In order to combat these insects, various methods have been recommended. The use of insecticides (LINDANE®, then MALATHION® and ethylene bromide) is currently being challenged because of the problems posed by the presence of residues of these products in food. In addition, resistance to these products has appeared in many target insects, which makes their use less and less effective. In order to replace these insecticides or limit their use, various methods have been proposed [for review, cf for example F. H. ARTHUR, J. Stored Prod. Res, 32, pp. 293-302, (1996)]. The methods which are currently the most developed are physical methods, such as the cooling of silos or storage under 002 or under nitrogen; these methods are, however, expensive and their use, which requires great technological sophistication, is delicate; they are therefore not applicable everywhere.

Another type of approach, which is the subject of much research, consists in producing transgenic plants expressing one or more gene(s) which confer(s) on them resistance against insect attack. However, this approach requires the availability of suitable genes, which must also be acceptable both for the environment and by consumers.

Most insects exhibit more or less strict food specificity; it is in this way that cereal seeds are attacked by grain weevils (Sitophilus oryzae, Sitophilus zeamais, Sitophiius granarius) which do not attack legume seeds; conversely, other pests, such as bruchid beetles, attack legumes but not cereals.

Previous studies by the inventors' team [DELOBEL and GRENIER, J. Stored Prod, Res, 29, pp. 7-14, (1993)] have shown that the three species of Sitophilus mentioned above can develop on chestnuts or acorns, but that, conversely, they die rapidly on split peas, this mortality being consecutive to the consumption of the peas by these weevils.

### BRIEF DESCRIPTION OF THE DRAWINGS

FIG. 1 shows a calibration curve calculated from each concentration of pea meal the time for 50% lethality (LT50) for the sensitive strain S.

FIG. 2 shows the cumulative mortality for adults of the sensitive strain S of Sitophilus oryzae, on pea (⋄) and on wheat (□) as a function of the feeding time in days.

FIG. 3 shows the mortality at 6 days of Sitophilus oryzae for balls containing various concentrations of pea meal; the resistant strain (R) and sensitive strain (S) are compared.

FIG. 4 shows the cumulative mortality of the Sitophilus oryzae weevils, resistant strain R or sensitive strain S measured after 5 (4A), 7 (4B), 14 (4C) and 20 (4D) days of feeding on cowpea (Vigna unguiculata) white (1) and red (2) variety bambora groundnut, lentil, French bean, mung bean, adzuki bean, broad bean, chickpea, and lupin.

FIG. 5 shows a chromatogram of the anion exchange chromatography described in Example 2.

FIG. 6 shows a chromatogram of the semipreparative reverse phase HPLC chromatography described in Example 2.

FIG. 7 shows the alignment of the sequence of one of the TP protein (SEQ ID NO:6), with those of pea PA1b protein (SEQ ID NO:7) and soybean leginsulin (SEQ ID NO:8).

FIG. 8 shows the results of testing the toxicity of the TP protein for the flour moth Ephestia kuehniellea (Lepidoptera) and for the aphid (Acyrthosiphon pisum).

FIG. 9 shows the results of testing of the aphid Acyrthosiphon pisum (Homoptera) fed on artificial medium containing various concentrations of the TP protein.

### DESCRIPTION OF THE INVENTION

The inventors have undertaken to investigate the toxic substance responsible for this mortality. It is, moreover, known that legumes contain several entomotoxic substances and that, in diverse species of insects for which legumes are toxic, there exist natural subpopulations which are more or less resistant to the toxicity of the legumes.

For example, in the case of grain weevils, a test carried out by the inventors' team on 90 strains of different geographical origins has shown that 4 strains belonging to the Sitophilus oryzae species include individuals capable of surviving to the adult stage on split peas; conversely, no strain having this ability has been revealed in the Sitophilus zeamais, or Sitophiius granarius species; the study of the genetic determinism of this resistance has shown that this property is monogenic, recessive and autosomal [GRENIER et al., Heredity, 79, pp. 15-23, (1997)].

The inventors have selected a strain of S. oryzae which is homozygous for this resistance gene, and have used this strain to investigate the toxic substance with respect to which the mechanism of resistance encoded by this gene is expressed.

The inventors have thus noted that this toxicity is associated with isoforms of a protein which has a sequence similar to that of the PA1b pea albumin (SEQ ID NO:7) described by HIGGINS et al. [J. Biol. Chem., 261 (24),pp. 11124-11130, (1986)], and which shows strong similarity (65% identity) with soybean leginsulin (SEQ ID NO:8) [WATANABE et al., Fur. J. Biochem., 15, pp. 224:1-167 72, (1994)]. No entomotoxic property had until now been associated with the PA1b protein (SEQ ID NO:7), with leginsulin or with other homologous proteins.

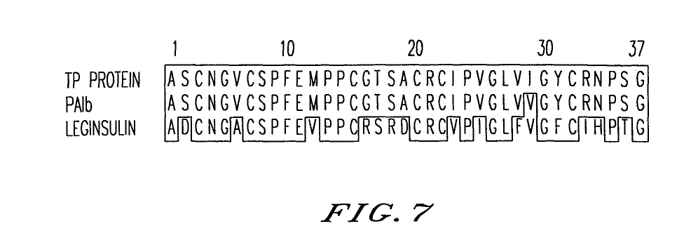

The alignment of the sequence of one of the isoforms of the protein purified by the inventors, with those of the pea PA1b protein (SEQ ID NO:7), published by HIGGINS et al., and of soybean leginsulin (SEQ ID NO:8), published by WATANABE et al., is represented in FIG. 7. These 3 sequences include in particular 6 cysteine residues which occupy conserved positions.

A subject of the present invention is the use, as an insecticide, of a polypeptide comprising a sequence which satisfies the following general formula (I):
X₁CX₂CX₃CX₄CX₅CX₆CX₇ (I) (SEQ ID NO: 1)
in which C represents a cysteine residue, X₁ represents an amino acid or a sequence of 2 to 10 amino acids, X₂ represents an amino acid or a sequence of 2 to 5 amino acids, X₃ represents a sequence of 4 to 10 amino acids, X₄ represents a sequence of 3 to 10 amino acids, X₅ represents an amino acid or a sequence of 2 to 4 amino acids, X₆ represents a sequence of 7 to 15 amino acids, and X₇ represents an amino acid or a sequence of 2 to 10 amino acids. Preferably, X₁ represents a dipeptide, X₂ represents a tripeptide, X₃ represents a heptapeptide, X₄ represents a tetrapeptide, X₅ represents an amino acid, X₆ represents a nonapeptide, and X₇ represents a pentapeptide.

Advantageously:
X₁ satisfies the sequence y₁y₂ in which y₁ and Y₂ each represent an amino acid chosen from alanine, serine, glycine and threonirie, or y₁ represents an amino acid chosen from alanine, serine, glycine and threonine, and Y₂ represents glutamic acid or aspartic acid; and/or
X₂ satisfies the sequence y₃y₄y₅ in which y₃ represents glutamine or asparagine, and y₄ and y₅ each represent an amino acid chosen from alanine, serine, glycine, threonine, valine, leucine, isoleucine and methionine; and/or
X₃ satisfies the sequence y₆y₇y₈y₉y₁₀y₁₁y₁₂ (SEQ ID NO:2) in which y₆ represents an amino acid chosen from alanine, serine, glycine and threonine, y₇, y₁₁ and Y₁₂ each represent proline, y₈ represents an amino acid chosen from phenylalanine, tryptophan and tyrosine, y₉ represents aspartic acid or glutamic acid, and y₁₀ represents an amino acid chosen from valine, leucine, isoleucine and methionine; and/or
X₄ satisfies the sequence y₁₃y₁₄y₁₅y₁₆ (SEQ ID NO:3), in which y₅ and y₁₆ each represent an amino acid chosen from alanine, serine, glycine and threonine, or y₁₄ represents an amino acid chosen from alanine, serine, glycine and threonine, y₁₃and y₁₅ each represent a basic amino acid, and y₁₆ represents aspartic acid or glutamic acid; and/or
X₅ represents a basic amino acid; and/or
X₆ satisfies the sequence y₁₇y₁₈y₁₉y₂₀y₂₁y₂₂y₂₃y₂₄y₂₅ (SEQ ID NO4), in which y₁₇, y₁₉, y₂₁ and y₂₃each represent an amino acid chosen from valine, leucine, isoleucine and methionine, y₁₈ represents proline, y₂₀and y₂₄ each represent an amino acid chosen from alanine, serine, glycine and threonine, y₂₂ represents an amino acid chosen from valine, leucine, isoleucine, methionine, phenylalanine, tryptophan and tyrosine, and y₂₅ represents an amino acid chosen from phenylalanine, tryptophan and tyrosine; and/or
X₇ satisfies the sequence y₂₆y₂₇y₂₈y₂₉y₃₀ (SEQ ID NO:5) in which y₂₆ represents a basic amino acid or an amino acid chosen from valine, leucine, isoleucine and methionine, y₂₇ represents asparagine or glutamine or a basic amino acid, y₂₈ represents proline, and y₂₉ and y₃₀ each represent an amino acid chosen from alanine, serine, glycine and threonine

According to one preferred embodiment of the present invention, the polypeptide used as an insecticide shows at least 40%, preferably at least 60%, homology with any one of the isoforms of a PA1b albumin.

For the purpose of the present invention, the term “PA1b albumin” is intended to mean not only any isoform of the pea PA1b protein, but also any protein of the same family which is present in other plants and which can especially be purified from seeds of legumes, in particular legumes of the Cesalpinaceae, Mimosaceae or Fabaceae family, or of the Meliaceae family, such as Khaya senegalensis

Polypeptides which can be used in accordance with the invention may be natural polypeptides, for example leginsulins of legumes, such as the soybean leginsulin (SEQ ID NO:8) described by WATANABE et al they may also be artificial polypeptides, the sequence of which is derived from that of a PA1b (SEQ ID NO:7) by adding, deleting or substituting a small number of amino acids It is possible to use, for example, polypeptides comprising a sequence which satisfies the general formula (I), or a portion of this sequence which corresponds to the region involved in the insecticidal activity This active peptide can optionally be fused, at its N-terminal end and/or at its C-terminal end, with another peptide sequence.

These polypeptides can be obtained by conventional methods, known per se, for example by peptide synthesis, or by genetic engineering, by expressing, in a suitable host cell, a sequence encoding the desired polypeptide. They can also, in the case of natural polypeptides, such as PA1b (SEQ ID NO:7) and leginsulin (SEQ ID NO:8), be purified from seeds of plants such as legumes or Meliaceae.

In accordance with the invention, the polypeptides comprising a sequence of general formula (I) (SEQ ID NO:1) can be used as the only active principle of an insecticide, or combined with one or more other active principles. They can be used in particular for combating insects which are pests for cereal seeds, and also for combating plant-feeding insects, such as the lepidoptera Mamestra brassicae or Ostrinia nubilalis or the coleoptera Chrysomeiidae, for instance Phaedon cochieariae or Curculionidae, for instance Anthonomus grandis, or combating phloem-feeding insects such as aphids.

Furthermore, the inventors have noted that the PA1b protein (SEQ ID NO:7) conserves its insecticidal activity for several years in dry seeds, and that this activity is not affected by heating to 100° C.

In addition, this protein is not toxic for humans or higher animals; it is present in the legumes which form part of their conventional diet.

The polypeptides of general sequence (I) (SEQ ID NO:1) are particularly suitable for protecting, especially during storage, seeds, flours or transformed products which are derived therefrom.

For the implementation of the present invention, the concentration of the polypeptide of sequence (I) (SEQ ID NO:1) in the product to be protected (plant, seeds or derived products) is generally from 10 μmol/kg to 100 mmol/kg (or from 10 μM to 100 mM), and advantageously from 50 μmol/kg to 10 mmol/kg (or from 50 μM to 10 mM).

According to one preferred embodiment of the present invention, the product to be protected as treated with a preparation comprising said polypeptide. This polypeptide can, for example, be in the form of a purified preparation or of an enriched fraction, which can in particular be obtained from seeds of plants which product said polypeptide naturally, or from cultures of cells which express a gene encoding this polypeptide.

According to another preferred embodiment of the present invention, a transgenic plant is produced which is transformed. with at least one gene encoding said polypeptide, and which expresses the latter in at least one of its tissues or organs.

The present invention also encompasses the transgenic plants produced in this way; advantageously, said plants are cereals.

These plants can be obtained by the conventional techniques of plant transgenesis, which are known per se.

It is thus possible to obtain, in a plant, ubiquitous expression and/or expression and/or overexpression in certain tissues or organs (for example in seeds) of a polypeptide of sequence (I) (SEQ ID NO:1), and as a result, to protect the plant, tissue or organ concerned against attacks by insects for which this polypeptide is toxic. In particular, the expression of a polypeptide of sequence (I) (SEQ ID NO:1) in the seeds makes it possible to protect them, even after harvest, as well as the transformed products and flours obtained from these seeds.

The present invention will be more clearly understood with the aid of the following description which refers to nonlimiting examples, describing the purification, and illustrating the insecticidal properties, of a legume PA1b albumin.

### EXAMPLE 1

### Demonstration of the Toxicity of Various Legu Als For Cereal Weevils

The toxicity of meals from various legumes was tested on weevils (Sitophilus oryzae) The experiments were carried out in parallel on wild-type animals (sensitive strain S), and on mutants surviving feeding on peas (resistant strain R).

The weevils (Sitophilus oryzae) are bred in a chamber regulated at 27.5° C. and 70% relative humidity. One-week-old adults are removed from these mass breeding colonies for the tests, For each test, experimentation is carried out on batches of 30 insects, and daily mortality is noted.

Balls of meal are kneaded with water, left to dry for 24 h and used for feeding the weevils. The gray wheat flour used is supplemented with various proportions of legume meal, sieved using a mesh size of 0.2 mm. The dose-response curves for weevil mortality were obtained using various doses of each meal to be tested. The results are processed using the “Toxicologie” [Toxicology] program [FEBVAY and RAHBE, “Toxicologie”, un programme pour l'analyse des courbes de mortalité par la méthode des probits sur MacIntosh [“Toxicology”, a program for analyzing mortality curves using the probits method on a MacIntosh computer], Cahiers Techn. INRA, 27, pp. 77-78 (1991)]. This program uses the transformation of the cumulative mortalities into probits, and determines the regression curve equation and the concentration for 50% lethality. These values are determined after exposure for 4 and 7 days.

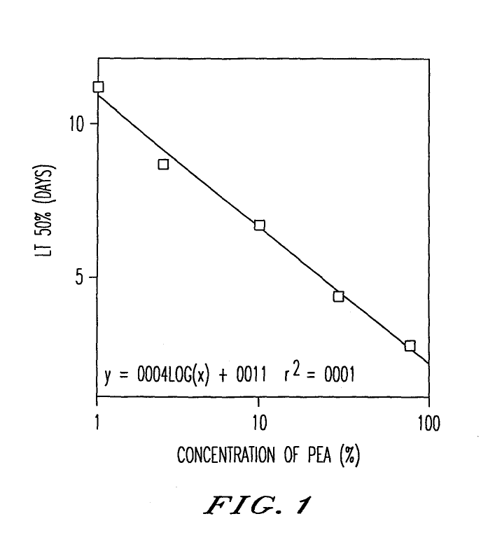

In addition, for each concentration of pea meal, the times for 50% lethality (LT50) for the sensitive strain S are also calculated. The calibration curve thus established makes it possible to determine, in the remainder of the experiments, for each meal or meal fraction tested, the equivalent concentration of pea meal (as % of pea in the wheat) This curve is given in FIG. 1.

### Pea Meal Toxicity

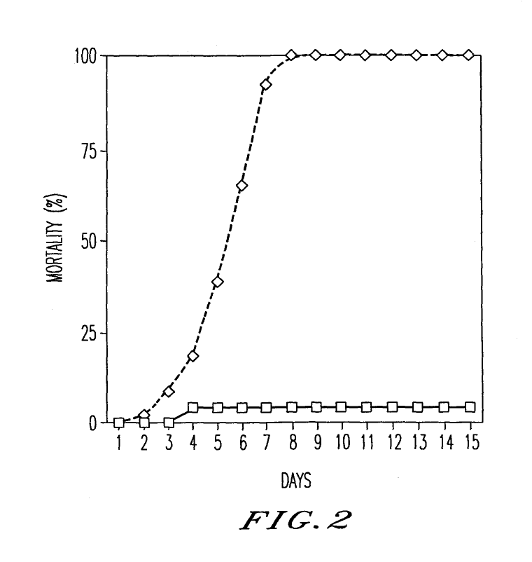

FIG. 2 shows the cumulative mortality for adults of the sensitive strain S of Sitophilus oryzae, on pea (⋄) and on wheat (□), as a function of the feeding time in days. These results show that the cereal weevils are rapidly killed on pea: in 8 days, between 90 and 100% of the adults are dead.

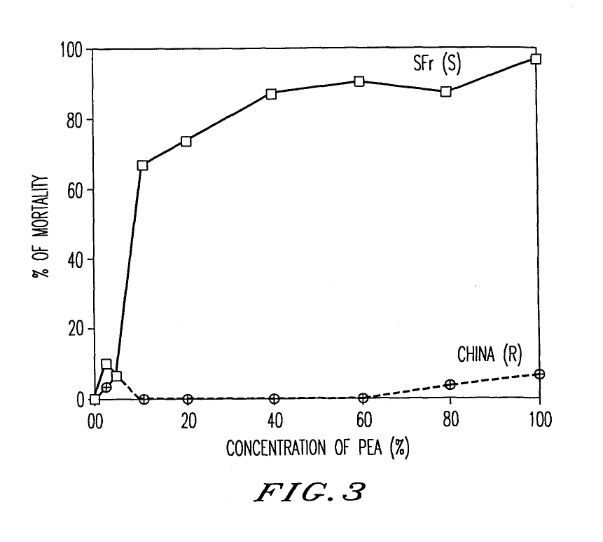

FIG. 3 shows the mortality at 6 days of Sitophilus oryzae, for balls containing various concentrations of pea meal; the resistant strain (R) and the sensitive strain (S) are compared. The dose/response curve thus established shows that, for the sensitive strain (S), from 10% of pea meal upward, 70% mortality s observed in 6 days (and 100% in 14 days) In the same period of time, the resistant strain (R) is not affected.

### Toxicity of Other Legume Meals

Among the legume seeds used in the human diet, 10 were tested for their action on the sensitive and resistant weevils.

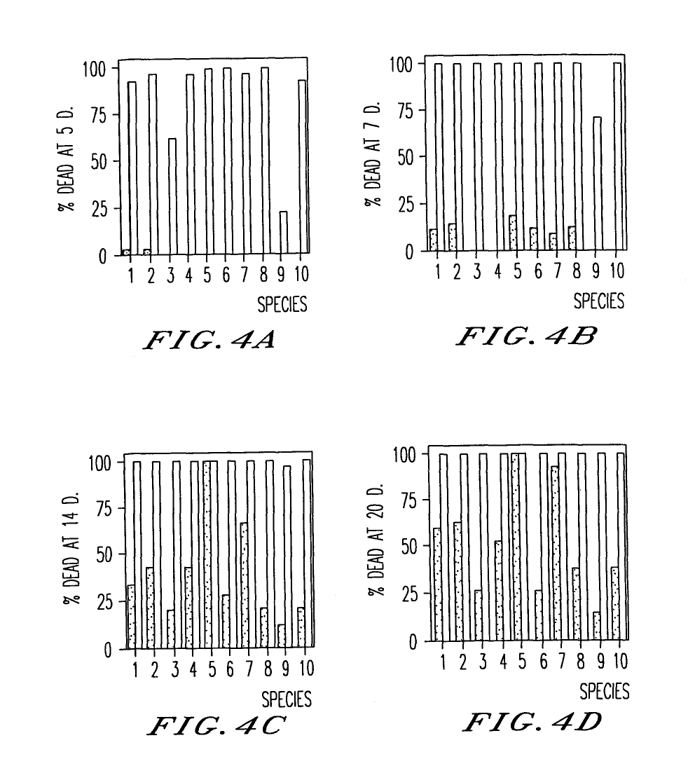

Balls containing 80% of legume meal and 20% of wheat flour were used. FIG. 4 illustrates the cumulative mortality of the Sitophilus oryzae weevils, resistant strain R [symbol] or sensitive strain S [symbol] measured after 5 (4A) 7 (4B), 14 (4C) and 20 (4D) days of feeding on cowpea (Vigna unguiculata) white (1) and red (2) variety bambora groundnut (3: Vigna subterranea), lentil (4: Lens esculenta), French bean (5: Phaseolus vulgaris), mung bean (6: Vigna radiata), adzuki bean (7: Vigna angularis), broad bean (8: Vicia faba), chickpea (9: Cicer arietinum), and lupin (10: Lupinus albus).

The results show that, at 7 days, all the legumes are toxic for the sensitive strain, even though Vigna subterranea and Cicer arietinurn have not yet killed all the insects which live thereon; conversely, the resistant strain shows no or very little mortality. It can therefore be concluded that the same mechanism causing the toxicity is present in all these legumes; this mechanism appears in particular to be predominant in Vigna subterranea, Vigna radiata and Cicer arietinum.

However, examination of the mortalities at 14 and 20 days on certain legumes reveals, for the resistant strain, higher or lower mortality which must, therefore, be attributed to other mechanisms; this is in particular the case on Phaseolus vulgaris and on Vigna anguiaris.

### EXAMPLE 2

### Purification And Identification of the Substance Responsible For the Toxicity In Peas

### Preparation of A Protein Fraction Enriched In Albumin (SRA1)

The fraction enriched in albumin is prepared on a pilot scale according to the protocol developed by CREVIEU et al, [Nahrung, 40 (5), pp. 237-244, (1996)].

The pea meal (10 kg) is mixed, with stirring, with 140 liters of acetate buffer (pH 49), the mixture is centrifuged at 7500 rpm and the supernatant is subjected to ultrafiltration on an MS membrane, at a temperature which does not exceed 25° C. The retentate is subject to diafiltration on the same membrane, the new retentate is centrifuged at 6000 rpm for 20 mm and the supernatant is lyophilized. The powder obtained (SRA1), which represents on average 1% of the mass used at the start, is used for the subsequent purifications.

At each step of the purification, the toxicity of the various fractions is determined according to the protocol described in Example 1 above.

### Anion Exchange Chromatography

10 g of SRA1 are suspended in 100 ml of a 60% methanol solution and stirred for 1 hour at 4° C. After centrifugation (30 mm, 9000 g, 4° C.), the supernatant is recovered and then the methanol present is removed in a rotary evaporator. The volume is then readjusted to 100 ml with water and a 1M Tris-HC1 buffer (pH 8.8) so as to obtain a final Tris-HCl concentration of 50 mM. The soluble proteins are fractionated by anion exchange chromatography on a DEAE SEPHAROSE FAST FLOW column (120×50 mm) The proteins adsorbed are eluted with a 50% concentration of buffer B (50 mM Tris-HCl, pH 8.8; 500 mM NaCl) in buffer A (50 mM Tris-HCl, pH 88). The elution flow rate is 20 ml/min and the fractions collected have a volume of 80 ml. The proteins are detected by absorption at 280 nm.

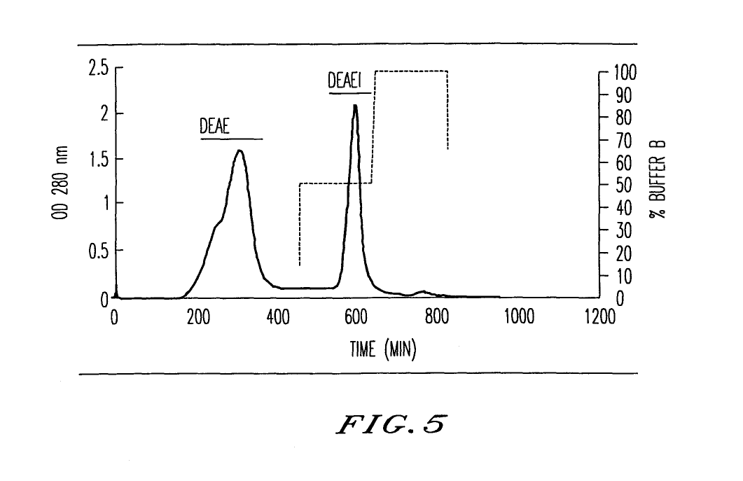

The chromatogram is shown in FIG. 5. The concentration of buffer B is indicated by the broken line. The 80 ml fractions corresponding to the peaks are pooled into two main fractions, DEAE NA and DEAE 1, indicated on the chromatogram by the horizontal lines. The nonadsorbed fraction (DEAF NA) contains all the toxicity.

This fraction is dialyzed against water for 72 hours and then lyophilized. Approximately 450 mg of the DEAE NA fraction are thus obtained.

### Semipreparative Reverse Phase HPLC Chromatography

The DEAE NA fraction obtained after anion exchange chromatography is fractionated by reverse phase HPLC (RP-HPLC) chromatography on a HYPERSIL column (250×10.5 mm) filled with C18-aliphatic-chain-grafted 5 μm 300 Å NUCLEOSIL. For each chromatography, 15 mg of proteins are loaded on to the column. The elution flow rate is 3 ml/min and the proteins are detected by absorption at 220 nm. The proteins are eluted with a gradient of buffer B (004% of trifluoroacetic acid in acetonitrile) in mixture A (0.04% of trifluoroacetic acid in water) according to the following sequence: t=0 mm, 40% of B; t=5 mm, 40% of B; t=17 mm, 48% of B; t=18 mm, 80% of B; and t=23 mm, 80% of B.

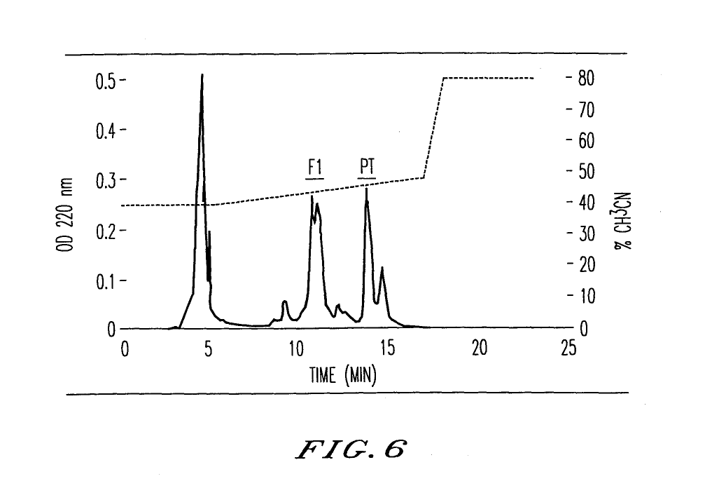

The chromatogram is illustrated in FIG. 6, The acetonitrile gradient is represented by the broken line. The toxicity is located only in the peaks Fl and TP; the fractions corresponding to these peaks which have been collected are represented on the chromatogram by horizontal lines.

Thirty successive chromatographies, corresponding to an injected amount of DEAF NA of 450 mg, were carried out. The fractions were pooled and then lyophilized after evaporating off the acetonitrile and the trifluoroacetic acid in a SPEED VAC, 4 mg of the TP fraction and 5 mg of El were thus obtained.

These fractions were then analyzed by reverse phase HPLC (RP-HPLC) chromatography.

### Reverse Phase HPLC Chromatograghy

The control of the purity of the proteins of the Fl and TP fractions is carried out by reverse phase HPLC chromatography on an INTERCHROM column (250×4.6 mm) filled with C18-aliphatic-chain-grafted 5 μm 100 Å NUCLEOSIL. The elution flow rate is 1 ml/min and the proteins are detected by absorption at 220 nm.

The proteins are eluted in 45 minutes with a linear gradient of 0 to 50% of mixture B (0.04% of trifluoroacetic acid in acetonitrile) in mixture A (0.04% of trifluoroacetic acid in water).

This analysis shows that the TP fraction contains only the toxic protein TP (SEQ ID NO:6). The Fl fraction is more complex and contains two major polypeptides.

### Characterization of the Proteins Present In the Fractions TP And Fl

The mass determinations were carried out by electrospray mass spectrometry (ES-MS). The mean masses calculated from 2 estimations are 3741.1 Da in the case of TP, and 3736 and 3941 Da for the polypeptides of the TF fraction. The number of cysteines free and involved in disulfide bridges was determined by alkylating the protein with iodoacetamide, before and after reduction, and comparing the retention times, by RP-HPLC, and the masses, by ES-MS, of the alkylated proteins with the native protein.

The alkylated nonreduced protein has both a retention time and a mass identical to that of the native protein. On the other hand, the protein which is reduced and then alkylated has a retention time which is clearly different from that observed for the native protein (30 mm instead of 42 mm) and a mass of 4089.9 Da.

It appears therefore that this protein contains 6 cysteines, which are all involved in 3 disulfide bridges.

### Complete Sequence of the TP Protein

The complete sequence of the TP protein (SEQ ID NO:6) was established. The mass calculated from the 37 residues of the protein is 374L4 Da, which is identical, give or take the measurement error, to that determined by mass spectrometry (3741.1 Da) for the native protein. The value calculated for the protein alkylated with iodoacetamide (4090 Da) is also equivalent to that obtained experimentally (4089.9 Da). These results demonstrate the absence of post-translational modifications (glycosylations, phosphorylations, etc.) of the protein.

The sequence of the TP protein (SEQ ID NO:6) shows very strong homology with that of the PA1b pea albumin (SEQ ID NO:7) [HIGGINS et al, J. Biol. Chem, 261 (24), pp. 11124-11130, (1986)]. The two sequences differ only by the replacement of the valine residue at position 29 in the TP protein (SEQ ID NO:6) with an isoleucine in PA1b (SEQ ID NO:7). Strong similarity (62% identity, 89% homology, determined with the aid of the MAC MOLLY program using the BLOSUM62 matrix) is also observed between the TP protein (SEQ ID NO:6) and soybean leginsulin (SEQ ID NO:8) [WATANABE et al., Eur. J. Biochem., 15, pp. 224:1-167-72, (1994)] In particular, the 6 cysteine residues, which play an essential role in the structure of the proteins, occupy conserved positions.

The comparison of these 3 sequences is shown in FIG. 7.

These results make it possible to conclude that the protein responsible for the resistance of pea to cereal weevils is similar to the PA1b protein (SEQ ID NO:7) described by HIGGINS. This protein is synthesized in the form of a 130-residue preproprotein (PAl) which undergoes post-translational maturation releasing the PA1b protein(SEQ ID NO:7) and a 53-residue protein named PA1a [HIGGINS et aL, J. Biol, Chem, 261 (24), pp. 11124-11130, (1986)].

Sequencing of the first 10 N-terminal residues of each of the toxic polypeptides of the Fl fraction was also carried out. The sequences obtained are identical to that of the N-terminal end of the TP protein. As, in addition, the masses of these polypeptides determined by ES-MS are very close to that of TP, it appears that these polypeptides represent isoforms of TP.

### EXAMPLE 3

### Activity And Stability of the Entomotoxic Proteins Extracted From Peas

Activity

The entomotoxic activity of the polypeptides of the TP fraction or of the Fl fraction was determined as described in Example 1 above; at the concentration of 1% in the wheat flour (3 mmol/kg), these polypeptides have a toxicity for the weevil which is equivalent to that of pure pea meal. A concentration of 60 tmol/kg is sufficient to prevent any infestation by the weevils.

Stability

The polypeptides of the TP fraction or of the Fl fraction, extracted from dried seeds stored for several years, conserve their entomotoxic activity. In addition, this activity is not affected by heating to 100° C.

Toxicity For Various Insects

The toxicity of the TP protein for the flour moth Ephestia kuehniellea (Lepidoptera) and for the aphid Acyrthosiphon pisum (Homoptera) was also tested.

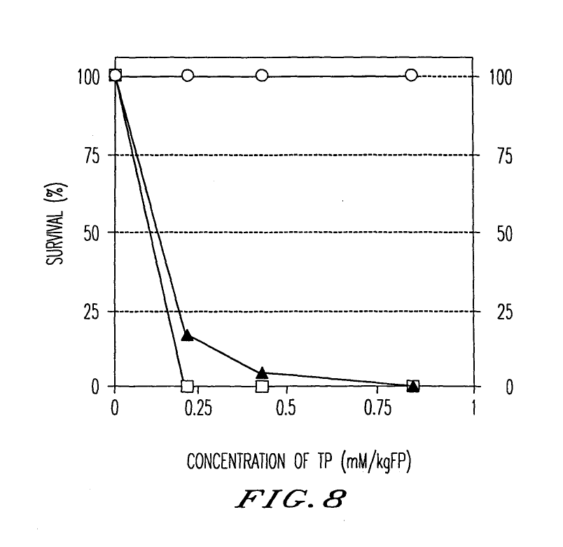

The tests on the flour moth were carried out on first and second stage Ephestia kuehniella larvae fed on wheat flour balls containing various concentrations of the TP protein (In mmol per kg of wheat flour). The results are shown in FIG. 8.

(◯=survival at 0 days;

▴=survival at 4 days;

□=survival at 10 days).

These results showed that this protein was very toxic, from the concentration of 0.25 mmol/kg upward.

The aphid Acyrthosiphon pisum (Homoptera) was fed on artificial medium containing various concentrations of the TP protein.

(□=3.3 μM;

◯=17 μM;

♦=46 μM;

◯=84 μM;

[symbol] =100 μM).

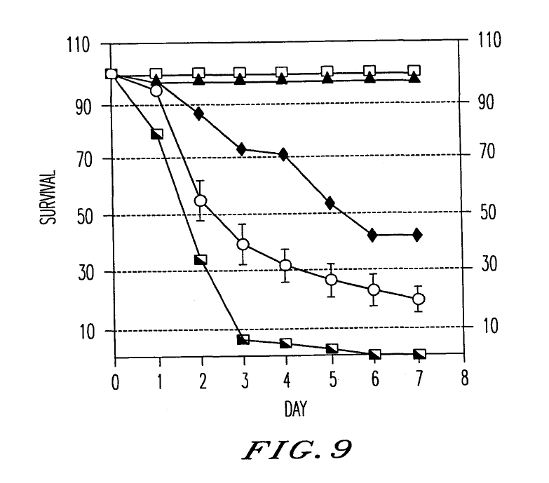

The results, which are shown in FIG. 9, show that considerable mortality appears from the concentration of 46 μmolar upwards this mortality being total at 100 μmolar.

1) The use, as an insecticide, of a polypeptide comprising a sequence which satisfies the following general formula (I):
X₁CX₂CX₃CX₄CX₅CX₆CX₇ (I)
in which C represents a cysteine residue, X₁ represents an amino acid or a sequence of 2 to 10 amino acids, X₂ represents an amino acid or a sequence of 2 to 5 amino acids, X₃ represents a sequence of 4 to 10 amino acids, X₄ represents a sequence of 3 to 10 amino acids, X₅ represents an amino acid or a sequence of 2 to 4 amino acids, X₆ represents a sequence of 7 to 15 amino acids, and X₇ represents an amino acid or a sequence of 2 to 10 amino acids.

2) The use as claimed in claim 1, characterized in that X₁ represents a dipeptide, X₂ represents a tripeptide, X₃ represents a heptapeptide, X₄ represents a tetrapeptide, X₅ represents an amino acid, X₆ represents a nonapeptide, and X₇ represents a pentapeptide.

3) The use as claimed in either of claims 1 and 2, characterized in that:
X₁ satisfies the sequence y₁y₂ in which y₁ and y₂ each represent an amino acid chosen from alanine, serine, glycine and threonine, or y₁ represents an amino acid chosen from alanine, serine, glycine and threonine, and y₂ represents glutamic acid or aspartic acid; and/or
X₂ satisfies the sequence y₃y₄y₅ in which y₃ represents glutamine or asparagine, and y₄ and y₅ each represent an amino acid chosen from alanine, serine, glycine, threonine, valine, leucine, isoleucine and methionine; and/or
X₃ satisfies the sequence y₆y₇y₈y₉y₁₀y₁₁y₁₂ in which y₆ represents an amino acid chosen from alanine, serine, glycine and threonine, y₇, y₁₁ and y₁₂ each represent proline, y₈ represents an amino acid chosen from phenylalanine, tryptophan and tyrosine, y₉ represents aspartic acid or glutamic acid, and y₁₀ represents an amino acid chosen from valine, leucine, isoleucine and methionine; and/or
X₄ satisfies the sequence y₁₃y₁₄Y₁₅y₂₆, in which y₁₃, y₁₄, y₁₅ and y₁₆ each represent an amino acid chosen from alanine, serine, glycine and threonine, or y₁₄ represents an amino acid chosen from alanine, serine, glycine and threonine, y₁₃ and y₁₅ each represent a basic amino acid, and y₁₆ represents aspartic acid or glutamic acid; and/or
X₅ represents a basic amino acid; and/or
X₆ satisfies the sequence y₁₇y₁₈y19y₂₀y21y₂₂y₂₃y₂₄y₂₅, in which y₁₇, y₁₉, y₂₁ and y₂₃ each represent an amino acid chosen from valine, leucine, isoleucine and methionine, y₁₈ represents proline, y₂₀ and y₂₄ each represent an amino acid chosen from alanine, serine, glycine and threonine, y₂₂ represents an amino acid chosen from valine, leucine, isoleucine, methionine, phenylalanine, tryptophan and tyrosine, and y₂₅ represents an amino acid chosen from phenylalanine, tryptophan and tyrosine; and/or
X₇ satisfies the sequence y₂₆y₂₇y₂₈y₂₉y₃₀ in which y₂₆ represents a basic amino acid or an amino acid chosen from valine, leucine, isoleucine and methionine, y₂₇ represents asparagine or glutamine or a basic amino acid, y₂₈ represents proline, and y₂₉ and y₃₀ each represent an amino acid chosen from alanine, serine, glycine and threonine.

4) The use as claimed in any one of claims 1 to 3, characterized in that the polypeptide used as an insecticide has at least 60% identity with any one of the isoforms of a PA1b albumin.

5) The use as claimed in claim 4, characterized in that said polypeptide is chosen from the group consisting of PA1b albumins and leginsulins.

6) The use as claimed in any one of claims 1 to 5, characterized in that said polypeptide is used for protecting cereal seeds, or products derived from them, against insect pests.

7) The use as claimed in any one of claims 1 to 5, characterized in that said polypeptide is used for protecting plants against insects which are pests for cereal grains.

8) The use as claimed in any one of claims 1 to 7, characterized in that said polypeptide is used at a concentration of 10 μmol/kg to 100 mmol/kg.

9) The use as claimed in claim 8, characterized in that said polypeptide is used at a concentration of 50 μmol/kg to 10 mmol/kg.

10) The use as claimed in any one of claims 1 to 9, characterized in that it comprises the treatment of the product to be protected with a preparation comprising said polypeptide.

11) The use as claimed in any one of claims 1 to 10, characterized in that it comprises the production of a transgenic plant which is transformed with at least one gene encoding said polypeptide, and which expresses the latter in at least one of its tissues or organs.

12) The use as claimed in claim 11, characterized in that said transgenic plant is a cereal.
